(19) United States
(12) Patent Application Publication
(10) Pub. No.: US 2025/0281761 A1
(43) Pub. Date: Sep. 11, 2025

(54) IMPLANTABLE CARDIAC DEVICE REMOTE RFID REPROGRAMMING SECURITY

(71) Applicant: Cardiac Pacemakers, Inc., St. Paul, MN (US)

(72) Inventors: Sean Thomas Horan, Maplewood, MN (US); Jonathan Bennett Shute, Eagan, MN (US); Bin Mi, Arden Hills, MN (US); Robert J. Parkinson, Woodbury, MN (US); Kevin G. Wika, Blaine, MN (US); Jonathan H. Kelly, Woodbury, MN (US)

(21) Appl. No.: 19/060,934

(22) Filed: Feb. 24, 2025

Related U.S. Application Data
(60) Provisional application No. 63/563,785, filed on Mar. 11, 2024.

(51) Int. Cl. A61N 1/372; A61N 1/378; G06F 21/44; G16H 40/67

(52) U.S. Cl. CPC A61N 1/37254; A61N 1/37223; A61N 1/3787; G06F 21/44; G16H 40/67

(57) ABSTRACT

Systems and methods to enable secure remote programming of an implantable medical device are disclosed, including implementing a patient-specific RFID communication protocol using first and second portions of configurable memory of an RFID circuit, separate from a communication circuit of the implantable medical device, to provide an initialization key using the first portion of configurable memory of the RFID circuit and receive an authorization sequence from the remote device using the second portion of configurable memory of the RFID circuit, wherein the implantable medical device or the RFID circuit is configured transition a state of the implantable medical device or the communication circuit of the implantable medical device based on the received authorization sequence stored in the second portion of configurable memory of the RFID circuit.

### CLAIM OF PRIORITY

This application claims the benefit of U.S. Provisional Application No. 63/563,785, filed on Mar. 11, 2024, which is hereby incorporated by reference in its entirety.

### TECHNICAL FIELD

This document relates generally to ambulatory medical devices and more particularly, but not by way of limitation, to systems and methods for secure remote reprogramming of implantable medical devices using Radio-Frequency Identification (RFID) technology.

### BACKGROUND

Medical devices, such as ambulatory, implantable, subcutaneous, wearable, insertable, or one or more other types of medical devices, can monitor, detect, or treat medical conditions of patients, including heart failure, atrial fibrillation, or one or more other medical conditions. Medical devices can include sensors to obtain or sense physiologic information from a patient and one or more circuits to detect one or more physiologic events or determine one or more physiologic metrics using the sensed physiologic information. Medical devices can include one or more communication circuits to transmit sensed physiologic information, detected physiologic events, or determine physiologic metrics to one or more remote or external devices separate from the medical device. Additionally, medical devices can be configured to provide electrical stimulation or one or more other therapies or treatments to the patient, such as to improve cardiac function, etc.

Remote programming or reprogramming of medical devices can reduce physician burden in adjusting or improving medical device utility, operation, and function. However, remote programming or reprogramming increases security risks associated with unauthorized or unsupervised access to protected memory and settings of the medical device that, in certain examples, can physically impact the patient, which may result in patient harm or violation of patient privacy.

### SUMMARY

Systems and methods to enable secure remote programming of an implantable medical device are disclosed, including implementing a patient-specific RFID communication protocol using first and second portions of configurable memory of an RFID circuit, separate from a communication circuit of the implantable medical device, to provide an initialization key using the first portion of configurable memory of the RFID circuit and receive an authorization sequence from the remote device using the second portion of configurable memory of the RFID circuit, wherein the implantable medical device or the RFID circuit is configured transition a state of the implantable medical device or the communication circuit of the implantable medical device based on the received authorization sequence stored in the second portion of configurable memory of the RFID circuit.

An example of subject matter (e.g., a medical device system configured to implement a patient-specific RFID communication protocol in an implantable medical device) may comprise means for providing an initialization key, means for receiving an authorization sequence from a remote device, and means for transitioning a state of the implantable medical device or a communication circuit of the implantable medical device based on the authorization sequence.

An example of subject matter (e.g., a medical device system) may comprise an implantable medical device comprising a control circuit configured to control operation of the implantable medical device, a communication circuit configured to communicate with a remote device, and an RFID circuit, separate from the communication circuit of the implantable medical device, wherein the RFID circuit comprises first and second portions of configurable memory, wherein the RFID circuit is configured to implement a patient-specific RFID communication protocol using the first and second portions of configurable memory.

In an example, which may be combined with any one or more examples described herein, to implement the patient-specific RFID communication protocol, the RFID circuit is optionally configured to provide an initialization key using the first portion of configurable memory of the RFID circuit and receive an authorization sequence from the remote device using the second portion of configurable memory of the RFID circuit.

In an example, which may be combined with any one or more examples described herein, the implantable medical device or the RFID circuit is optionally configured transition a state of the implantable medical device or the communication circuit of the implantable medical device based on the authorization sequence stored in the second portion of configurable memory of the RFID circuit.

In an example, which may be combined with any one or more examples described herein, to transition the state of the implantable medical device optionally includes to enable communication between the remote device and the communication circuit of the implantable medical device based on the authorization sequence stored in the second portion of configurable memory of the RFID circuit.

In an example, which may be combined with any one or more examples described herein, the RFID circuit is optionally configured to authenticate the remote device for secure remote programming of the implantable medical device using the first and second portions of configurable memory of the RFID circuit without activating the communication circuit of the implantable medical device.

In an example, which may be combined with any one or more examples described herein, the RFID circuit is optionally a passive or semi-passive RFID circuit configured to receive power from the remote device separate from the implantable medical device.

In an example, which may be combined with any one or more examples described herein, the implantable medical device optionally includes a power source configured to supply power to the control circuit and the communication circuit, wherein the RFID circuit is a passive RFID circuit configured to receive power from the remote device separate from the implantable medical device and does not receive power from the power source of the implantable medical device.

In an example, which may be combined with any one or more examples described herein, the RFID circuit optionally comprises a memory circuit comprising a limited amount of configurable memory, wherein the limited amount of configurable memory consists of the first and second portions of configurable memory.

In an example, which may be combined with any one or more examples described herein, the implantable medical device optionally comprises a data bus configured to enable communication between the communication circuit of the implantable medical device and the control circuit of the implantable medical device, the RFID circuit comprises an RFID control circuit and an internal data bus between the RFID control circuit and the configurable memory of the RFID circuit, and the RFID circuit comprises an RFID antenna, separate from an antenna of the communication circuit, configured to receive energy to power the internal data bus of the RFID circuit.

In an example, which may be combined with any one or more examples described herein, the RFID control circuit is optionally configured to control activation of the data bus between the communication circuit of the implantable medical device and the control circuit of the implantable medical device based on the authorization sequence stored in the second portion of configurable memory of the RFID circuit.

In an example, which may be combined with any one or more examples described herein, the initialization key is optionally a random sequence provided by the RFID circuit, wherein the authorization sequence is a patient-specific passkey generated by an authentication circuit separate from the remote device and the implantable medical device or the RFID circuit is configured to transition the state of the implantable medical device or the communication circuit of the implantable medical device based on the patient-specific passkey generated by the authentication circuit and stored in the second portion of configurable memory of the RFID circuit.

In an example, which may be combined with any one or more examples described herein, the implantable medical device is optionally configured transition a state of the communication circuit of the implantable medical device based on the authorization sequence stored in the second portion of configurable memory of the RFID circuit.

In an example, which may be combined with any one or more examples described herein, the RFID circuit is optionally configured transition a state of the implantable medical device based on the authorization sequence stored in the second portion of configurable memory of the RFID circuit.

In an example, which may be combined with any one or more examples described herein, to implement the patient-specific RFID communication protocol, the RFID circuit is optionally configured to detect, at a first time, a wake-up signal from the remote device, activate an internal data bus of the RFID circuit, receive, at a second time subsequent to the first time, an identifier of the remote device using one of the first or second portions of configurable memory of the RFID circuit, provide, at a third time subsequent to the second time, the initialization key using the first portion of configurable memory of the RFID circuit, receive, at a fourth time subsequent to the third time, the authorization sequence from the remote device using the second portion of configurable memory of the RFID circuit, and authenticate, at a fifth time subsequent to the fourth time, the remote device for secure remote programming of the implantable medical device using the communication circuit of the implantable medical device based on the authorization sequence and the initialization key.

In an example, which may be combined with any one or more examples described herein, to authenticate the remote device optionally comprises to perform multi-factor authentication of a clinician providing programming instructions for secure remote programming of the implantable medical device, perform second-level approval by a second clinician of the programming instructions for secure remote programming of the implantable medical device, perform patient approval of the clinician providing programming instructions or programming instructions for secure remote programming of the implantable medical device, or combinations or permutations thereof.

An example of subject matter (e.g., a medical device system) may comprise an ambulatory medical device comprising a control circuit configured to control operation of the ambulatory medical device and a communication circuit configured to communicate with a remote device, wherein the ambulatory medical device is configured to authenticate the remote device for secure remote programming of the ambulatory medical device using the communication circuit of the ambulatory medical device, wherein to authenticate the remote device comprises to perform at least one of multi-factor authentication of a clinician providing programming instructions for secure remote programming of the ambulatory medical device, second-level approval by a second clinician of the programming instructions for secure remote programming of the ambulatory medical device, or patient approval of the clinician providing programming instructions or programming instructions for secure remote programming of the ambulatory medical device. The ambulatory medical device can optionally transition a state of the ambulatory medical device or the communication circuit of the ambulatory medical device based on the authentication.

An example of subject matter (e.g., a method) may comprise implementing a patient-specific RFID communication protocol using first and second portions of configurable memory of an RFID circuit of an implantable medical device, wherein the implantable medical device comprises a control circuit configured to control operation of the implantable medical device and a communication circuit configured to communicate with a remote device, wherein the communication circuit is separate from the RFID circuit.

In an example, which may be combined with any one or more examples described herein, implementing the patient-specific RFID communication protocol optionally comprises providing an initialization key using the first portion of configurable memory of the RFID circuit, receiving an authorization sequence from the remote device using the second portion of configurable memory of the RFID circuit, and transitioning a state of the implantable medical device or the communication circuit of the implantable medical device based on the authorization sequence in the second portion of configurable memory of the RFID circuit.

In an example, which may be combined with any one or more examples described herein, transitioning the state of the implantable medical device optionally includes enabling communication between the remote device and the communication circuit of the implantable medical device based on the authorization sequence stored in the second portion of configurable memory of the RFID circuit.

In an example, any one or more examples described herein may further optionally comprise authenticating the remote device for secure remote programming of the implantable medical device using the first and second portions of configurable memory of the RFID circuit without activating the communication circuit of the implantable medical device.

In an example, any one or more examples described herein may further optionally comprise receiving power from the remote device separate from the implantable medical device to power the RFID circuit, wherein the RFID circuit is a passive or semi-passive RFID circuit.

In an example, any one or more examples described herein may further optionally comprise supplying power to the control circuit and the communication circuit using a power source of the implantable medical device and receiving power from the remote device separate from the implantable medical device to power the RFID circuit, wherein the RFID circuit is a passive RFID circuit configured that does not receive power from the power source of the implantable medical device.

In an example, which may be combined with any one or more examples described herein, the RFID circuit optionally comprises a memory circuit comprising a limited amount of configurable memory, wherein the limited amount of configurable memory consists of the first and second portions of configurable memory.

In an example, any one or more examples described herein may further optionally comprise enabling communication between the communication circuit of the implantable medical device and the control circuit of the implantable medical device using a data bus of the implantable medical device, enabling communication between an RFID control circuit of the RFID circuit and the configurable memory of the RFID circuit using an internal data bus of the RFID control circuit, and receiving energy to power the internal data bus of the RFID circuit using an RFID antenna of the RFID circuit, separate from an antenna of the communication circuit.

In an example, any one or more examples described herein may further optionally comprise controlling, using the RFID control circuit, activation of the data bus between the communication circuit of the implantable medical device and the control circuit of the implantable medical device based on the authorization sequence stored in the second portion of configurable memory of the RFID circuit.

In an example, a system or apparatus may optionally combine any portion or combination of any portion of any one or more of the examples described herein, may optionally combine any portion or combination of any portion of any one or more of the examples described herein to comprise “means for” performing any portion of any one or more of the functions or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example medical device system including an implantable medical device and a remote device.

FIG. 2 illustrates an example RFID circuit.

FIG. 3 illustrates an example method of implementing a patient-specific RFID communication protocol using an RFID circuit of an implantable medical device.

FIG. 4 illustrates an example medical device system.

FIG. 5 illustrates an example patient management system and portions of an environment in which the system may operate.

FIG. 6 illustrates a block diagram of an example machine upon which any one or more of the techniques (e.g., methodologies) discussed herein may perform.

### DETAILED DESCRIPTION

The present inventors have recognized, among other things, systems and methods to provide low-power, secure remote programming or reprogramming of implantable or other ambulatory medical devices by a remote device (e.g., a remote programmer, a cellular phone, a mobile device, or one or more other external electronic device executing a programming application, etc.) using Radio-Frequency Identification (RFID) technology.

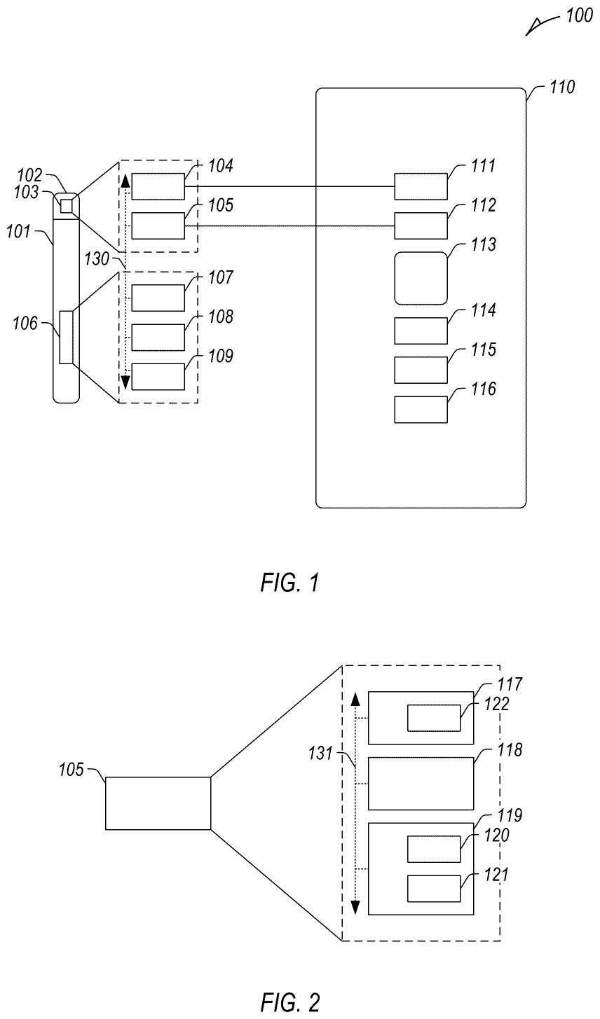

FIG. 1 illustrates an example medical device system 100 including an implantable medical device 101, such as an insertable cardiac monitor (ICM) or other implantable medical device, and a remote device 110, such as a cellular phone, a medical device programmer, or one or more other portable or other electronic device. In various examples, the remote device 110 can be one or more component of the external system 505 described in FIG. 5 (e.g., the external device 506, the remote device 508, etc.).

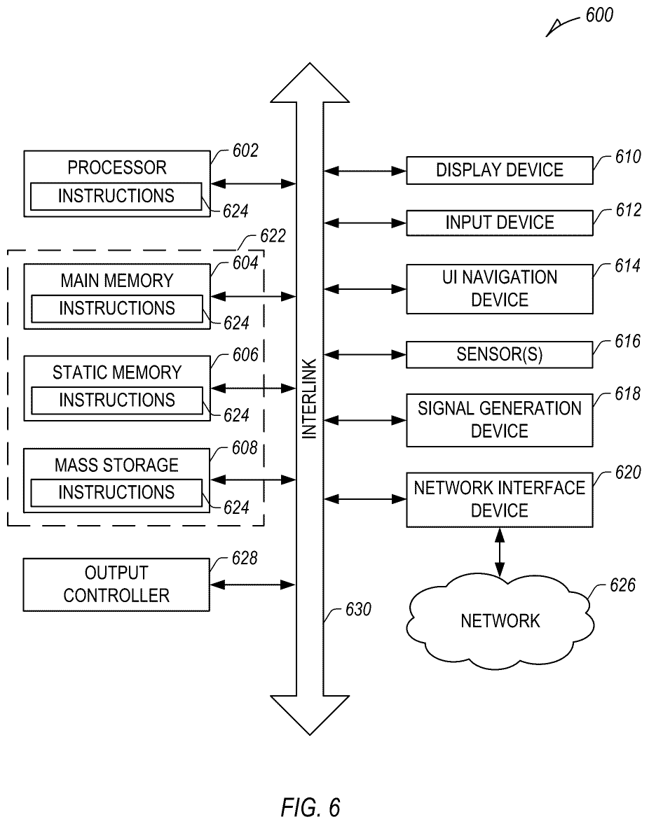

The implantable medical device 101 can comprise a housing (or “can”) composed of a biocompatible material, such as titanium, etc., and can include a header 102 composed of a biocompatible or implantable grade polymer. In certain examples, one or both of the housing and the header 102 can include one or more electrodes. The header can include one or more circuits 103, such as one or both of a communication circuit 104 of the implantable medical device 101 (or one or more components thereof (e.g., an antenna, etc.)) and an RFID circuit 105 (or one or more components thereof (e.g., an RFID antenna, etc.), separate from the communication circuit 104. The housing of the implantable medical device 101 includes additional circuits 106, such as a control circuit 107, a memory circuit 108, a power source 109 (e.g., a battery), etc. Additionally, the implantable medical device 101 can include a data bus 130 to enable communication between different components of the implantable medical device (e.g., similar to the interlink 630 illustrated in FIG. 6). In certain examples, the data bus 130 can include different portions, such as a first portion between one or more sensors of the implantable medical device and the control circuit 107 or the memory circuit 108, and a second portion between the communication circuit 104 and the control circuit 107 or the memory circuit 108, etc.

The remote device 110 includes a remote communication circuit 111 configured to communicate with the communication circuit 104 of the implantable medical device 101, a remote RFID circuit 112 (e.g., an RFID reader or interrogator, etc.) configured to communicate with the RFID circuit 105 of the implantable medical device 101, a magnet 113 (e.g., to wake up or initiate communication with the implantable medical device 101 or the RFID circuit 105), and other circuit components, such as a remote control circuit 114, a remote memory circuit 115, a remote power source 116 (e.g., a battery), etc. In certain examples, the magnet 113 can be a component of a protective case of the remote device 110.

FIG. 2 illustrates an example of the RFID circuit 105 of FIG. 1, including an RFID antenna 117, an RFID control circuit 118, and an RFID memory circuit 119. Although the RFID circuit 105 and the communication circuit 104 of the implantable medical device 101 can include similar components (e.g., an antenna, a control circuit, a memory circuit, a data bus, etc.), the RFID circuit 105 is configured to perform a different type of communication through the RFID memory circuit 119 than that performed by the communication circuit 104.

The most power intensive operation of many implantable medical devices is traditional wireless communication of data into and out of implantable medical devices by their respective communication circuits, representing the largest current draw on limited power supplies of such devices. Traditional wireless communication is represented in FIG. 1 by the communication circuit 104, and typically includes one or more different types of communication, such as: Medical Implant Communication Service (MICS) (402-405 MHz frequency with a range of 2 m and data rate of 250 kbps); Bluetooth® or Bluetooth® Low Energy (BLE) (2.4-2.483 GHz frequency with a range up to 10 m and data rates up to 2 Mbps); or Near Field Communication (NFC) (13.56 MHz with a range up to 10 cm, typically 4 cm or less, and data rates up to 424 kbps). Power for such traditional wireless communication is generally provided by the power source 109 of the implantable medical device 101.

In contrast, the RFID circuit 105 is configured to enable communication (e.g., 13.56 MHz) with a maximum communication range measured in inches, up to 1 m, and data rates limited by configurable memory servicing the RFID circuit 105, and in certain examples powered by or at least in part powered by a remote device 110 seeking communication therewith. Communication with the remote device 110 through the communication circuit 104 requires that the data bus 130 of the implantable medical device 101 (e.g., at least the second portion of the data bus 130 between the communication circuit 104 and the control circuit 107 or the memory circuit 108 of the implantable medical device 101) to be in a powered or active state, where the implantable medical device 101 is an active or a communication mode, including communication between the control circuit 107, the memory circuit 108, and the communication circuit 104. In contrast, communication through the RFID circuit 105 does not require that the communication circuit 104 or the data bus 130 of the implantable medical device 101 (or at least a portion of the data bus 130 associated with the communication circuit 104) be active in the implantable medical device 101, such that the implantable medical device 101 can be in one or more low-power states with one or more components in an inactive state (e.g., the data bus 130, the communication circuit 104, etc.), with the communication circuit 104 inactive or operating at a lower power level than required during polling or active communication. Accordingly, using the RFID circuit 105 for communication, such as daily communication, or to initialize other communication with the implantable medical device 101, instead of using the communication circuit 104 for such possibly denied or unauthorized communication attempts, can represent significant power savings to the implantable medical device 101 over traditional techniques.

The RFID circuit 105 can include an active, passive, or semi-passive RFID circuit to implement a patient-specific RFID communication protocol, such as to initialize communication (e.g., performing handshaking, discovery, etc.) between the remote device 110 and the communication circuit 104 of the implantable medical device 101. Initializing communication between the implantable medical device 101 and the remote device 110 using the RFID circuit 105 can significantly reduce the amount of power associated with confirming or validating authorization associated with communication by the remote device 110, such as in contrast to performing initialization using the communication circuit 104 of the implantable medical device 101 alone. The RFID memory circuit 119 can be restricted by default to a read-only mode, preventing unauthorized write access. A patient-specific RFID communication protocol can be performed by the remote device 110 or between the remote device 110 and the RFID circuit 105 to enable, and in certain examples power, the RFID circuit 105 or one or more aspects of the RFID circuit, such as the RFID control circuit 118, the RFID memory circuit 119, or one or more other components, for example, to allow data to be written to or read from the RFID memory circuit 119 or between the RFID circuit 105 and one or more other components of the implantable medical device 101, such as additional circuits 106 of the implantable medical device 101, etc.

In an example, the RFID circuit 105 can remain in a dormant or low-power state (e.g., idle) until receiving a wake-up signal. In an example, the wake-up signal can include a detected proximity by the RFID circuit 105 or one or more other components (e.g., pic field sensors) of the implantable medical device 101 to a magnetic field, such as a magnetic field strength above a threshold (e.g., from the magnet 113 of the remote device 110, from an electromagnetic field emitted by the remote device 110, or combinations thereof, etc.). Traditionally, such wake-up would enable the communication circuit 104 of the implantable medical device 101, or the communication circuit 104 of the implantable medical device 101 would poll at regular periods for potential wireless connections, each requiring substantial resources by the power source 109 of the implantable medical device 101 that often results in no communication session at all (e.g., false trigger, unauthorized remote device, etc.). With the RFID circuit 105, however, receiving the wake-up signal can merely activate the RFID circuit 105 to receive or transmit information, without impacting the remaining components of the implantable medical device 101, such as the communication circuit 104, the data bus 130 of the implantable medical device 101, etc. In an example, the RFID circuit 105 can include an internal data bus 131 specific to the components of the RFID circuit 105 (e.g., similar to the interlink 630 illustrated in FIG. 6, although internal to the RFID circuit 105), separate from the data bus 130 of the implantable medical device 101, and the wake-up signal can activate the internal data bus 131 of the RFID circuit 105 without impacting the data bus 130 of the implantable medical device 101.

In a passive RFID circuit, energy from the electromagnetic field emitted by the remote device 110 can be converted into energy, such as by converting a changing magnetic field of a radio-frequency (RF) signal to current to power components of the RFID circuit 105, such as using the RFID antenna 117 and power components 122 (e.g., a rectifier and a voltage regulator, etc.), without receiving separate power from the implantable medical device 101 (or in certain examples in addition to power from the implantable medical device 101, though still reducing the overall power required by the power source 109 of the implantable medical device 101). The idle/standby power of the passive RFID circuit, and in certain examples active or semi-active RFID circuits, can be orders of magnitude lower than the communication circuit 104 of the implantable medical device 101. In certain examples, proximity (a distance within a threshold in inches) of the RFID circuit 105 or the implantable medical device 101 to the remote device 110 (or the magnet 113) can be required to provide an additional level of security, including consent of the patient to physical proximity to their body including a location of the implantable medical device 101 and the RFID circuit 105. Accordingly, in addition to the RFID antenna 117, the RFID circuit 105 can additionally include one or more circuits or components, such as the power components 122, configured to convert received energy into current to power the RFID circuit 105.

For example, the magnet 113 can be detected by the implantable medical device 101 or the RFID circuit 105, such as using one or more sensors (e.g., a magnometer, a Hall Effect Sensor, a switch, such as a reed switch, sensitive to a magnetic field, etc.), and used to enforce a proximity requirement of the remote device 110 to the patient, the implantable medical device 101, or the RFID circuit 105 of the implantable medical device (e.g., contact or near-contact with the epidermis of the patient or clothing of the patient over the implantable medical device 101, such as within 6 inches of the RFID circuit 105 of the implantable medical device 101, within 4 inches of the RFID circuit 105 of the implantable medical device 101, or in certain examples less), during initialization of communication with or remote programming or reprogramming of the implantable medical device 101 (or in certain examples throughout communication or programming or reprogramming of the implantable medical device 101). In other examples, the amount of power provided to the RFID circuit 105 from the remote device 110 can be used to determine and enforce a proximity determination, as the amount or magnitude of power transferred by a magnetic field varies with distance from the source by the inverse square law.

In an example, the RFID memory circuit 119 can be configurable or programmable (e.g., memory able to be written to or read from by a remote user). The amount of configurable memory in different RFID circuits can vary significantly, such as between 0 bits and 8 kilobytes or greater. Some RFID circuits have no user memory, where user memory is memory outside of Electronic Product Code (EPC) memory (e.g., an identifier of the product to which the RFID circuit is coupled), Tag Identifier (TID) memory (e.g., an identifier of the RFID circuit), or, in certain examples, reserved memory or lock bits that control access and operation of the RFID circuit. Other RFID circuits include additional user memory for storage of other information.

Although operable with larger amounts of user memory, one example described herein includes the RFID circuit 105 being a passive RFID circuit (or a semi-passive RFID circuit still at least partially powered by energy from the remote device) consisting of a limited amount of configurable memory, in certain examples, only enough memory to store first and second sequences of data in respective first and second portions 120, 121.

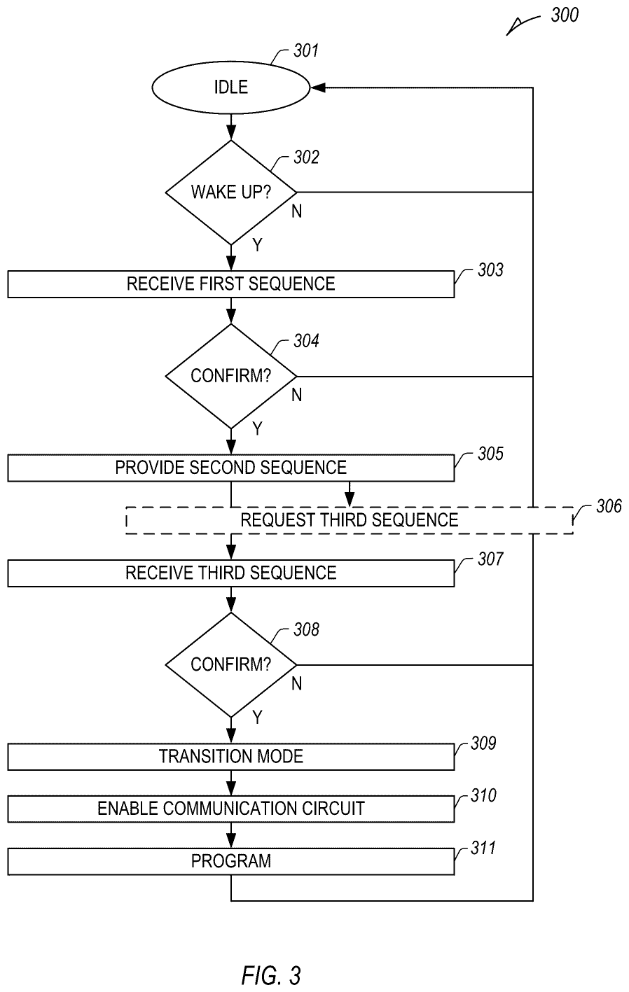

FIG. 3 illustrates an example method 300 of implementing a patient-specific RFID communication protocol using an RFID circuit of an implantable medical device.

At step 301, the RFID circuit can be in an idle state drawing little or no power. In certain examples, the implantable medical device or components thereof can be in an idle state, such as a communication circuit of the implantable medical device separate from the RFID circuit, a data bus in the implantable medical device between a control circuit, a memory circuit, and the communication circuit, etc. In other examples, the idle state can include a dormant or low-power state defined or limited by a maximal allowable idle state power draw on a power source of the implantable medical device or a state or status of one or more components of the implantable medical device.

If a wake-up signal is detected at step 302, such as by detecting a magnetic field strength above a threshold, etc., power can be applied to the RFID circuit, in certain examples using power from a detected magnetic field, to enable the RFID circuit to receive a first sequence (e.g., an identifier of the remote device, etc.) from a remote device at step 303 and storing (e.g., writing) the received first sequence in a first portion of a memory circuit of the RFID circuit. In an example, applying power to the RFID circuit can include activating an internal data bus of the RFID circuit. The first sequence can include information about the remote device providing the first sequence. If wake up is not detected at step 302, process can return to step 301.

In certain examples, continuity of the wake-up signal can be required throughout one or more other steps of the method 300, where a lack of continuity can trigger return to step 301.

At step 304, one of the RFID circuit or the control circuit of the implantable medical device can confirm the received first sequence, such as by determining, based on the received and stored first sequence, that the device that provided the wake-up signal is authorized or is a type that is authorized to communicate with the RFID circuit or the implantable medical device. In certain examples, to make such determination, the data bus (e.g., the internal communication bus) of the implantable medical device can be enabled, such as to perform confirmation using the control circuit of the implantable medical device. However, the communication circuit of the implantable medical device can remain in an off or idle state. In other examples, the RFID circuit can perform such confirmation without enabling the data bus of the implantable medical device. If the received first sequence is confirmed at step 304, process can continue to step 305. If the received first sequence is not confirmed at step 304, process can return to step 301.

At step 305, one of the control circuit of the implantable medical device or the RFID circuit can provide a second sequence in a second portion of the memory circuit of the RFID circuit. In an example, the second sequence can include an initialization key of the RFID circuit or the implantable medical device, or in certain examples, a random sequence to be provided to an authentication circuit.

At step 306, the remote device can receive (e.g., read) the second sequence from the second portion of the memory circuit, and optionally request a third sequence (e.g., an authentication sequence) from an authentication circuit, such as by providing the received second sequence to the authentication circuit and receiving the third sequence in response thereto. The third sequence can include, in certain examples, a patient-specific passkey generated in response to authorization by one or both of the patient or a clinician of the patient having knowledge of the patient, the patient's history, and the implantable medical device. In certain examples, the remote device can provide information about the remote device or a user of the remote device to the authentication circuit to determine if the remote device is authorized to enable communication with the RFID circuit or the implantable medical device.

In an example, the authentication circuit can be a component of the remote device or a component separate from the remote device (e.g., one or more of the components of the external system 505, such as the external device 506, the remote device 508, etc.), and can receive the second sequence (e.g., the initialization key) from the remote device, and in certain examples information about the remote device or the user of the remote device, and determine, using information from a clinician or a user associated with implantable medical device, whether the remote device or the user associated with the remote device is authorized to communicate with or program the implantable medical device coupled to the RFID circuit. In certain examples, a clinician or other caregiver associated with the patient can authorize specific reprogramming, data output, or other actions for the implantable medical device, such as by one or more users, type of remote device, or specific remote device. The authentication circuit can receive and store such authorizations. Upon determining that the remote device is authorized to communicate with the implantable medical device, and permissions associated with such authorization (e.g., a type of communication or programming, such as read-only, read/write, parameter settings available for adjustment, etc.), the authentication circuit can provide the third sequence to the remote device.

The third sequence can include information that can, once presented to the RFID circuit or the implantable medical device, indicate a level of authorization or permission with respect to the patient or the implantable medical device. In certain examples, the third sequence can interact with the second sequence in a specific manner to provide instructions to the implantable medical device. Security can be provided based on one or more predefined interactions between such sequences. In certain examples, depending on the authorization or access associated with the third sequence from the authentication circuit, the implantable medical device may operate in one of a plurality of different modes, such as one or more read-only modes, for example, enabling/powering the communication circuit of the implantable medical device to provide data output without allowing write access to the implantable medical device or alteration of programming parameters, or one or more read/write modes, enabling programming of the implantable medical device, including altering operation modes, programming parameter settings, etc.

For example, different levels can include, among others: read-only access to an implantable medical device status; read-only access of parameter settings; read-only access to patient information (e.g., identity, patient physiologic information, etc.); partial-write access to specific parameter settings that do not physically impact the patient; partial-write access to one or more parameter settings that may impact the patient; full read-write access; etc. Different levels of authorization are described in the commonly assigned Wika et al. U.S. Application Ser. No. 63/555,625 entitled “BLE PROGRAMMER INSTRUCTION CLASSES,” which is hereby incorporated by reference in its entirety, including its disclosure of different programming instructions and classes and communication. The third sequence provided to the remote device can be specific to one or more of these levels of authorization or permissions, and in certain examples specific to one of the first or second sequences, etc.

Responsive to receiving the third sequence, the remote device can write the third sequence to the first portion of the memory circuit of the RFID circuit, in certain examples still without enabling the communication circuit of the implantable medical device (e.g., separate from scheduled upload of information to one or more remote devices, etc.).

At step 307, the third sequence can be received by the RFID circuit, such as, for example, storing (e.g., writing) the received third sequence in the first portion of the memory circuit of the RFID circuit.

At step 308, one of the RFID circuit or the control circuit of the implantable medical device can confirm the received third sequence, such as by determining, based on the received and stored third sequence, that the device that provided the third sequence is authenticated by the authentication circuit to communicate with the RFID circuit or the implantable medical device, and the appropriate level of communication, including programming or reprogramming the implantable medical device or changing or altering one or more parameter settings or modes of the implantable medical device. In certain examples, to make such determination, the data bus of the implantable medical device can be enabled, such as to perform confirmation using the control circuit of the implantable medical device. In other examples, the RFID circuit can perform such confirmation and only enable the data bus of the implantable medical device following successful confirmation.

In an example, to perform confirmation, the implantable medical device can receive information from the RFID circuit, including the third sequence (and in certain examples the first or second sequences), confirm the third sequence (in certain examples combined with one or both of the first or second sequences), determine the representative authorization or permission associated therewith, and perform one or more operations consistent with the associated authorization or permission.

If the received third sequence is confirmed at step 308, process can continue, for example, to step 309, step 310, step 311, etc. For example, upon a confirmed third sequence, the communication circuit of the implantable medical device can be enabled to negotiate communication with the remote device (e.g., enable discovery, pairing, connection, etc.). In such context, enabled can include triggering the communication circuit of the implantable medical device to wake up or change from a passive or inactive state to an active state in response to the confirmation at step 308. In other examples, the memory circuit of the RFID circuit, upon a confirmed third sequence, can be used to provide information for pairing and connection with the communication circuit of the implantable medical device. If the received third sequence is not confirmed at step 308, process can return to step 301.

At step 309, one of the control circuit of the implantable medical device or the RFID circuit can provide a signal to transition a mode of the RFID circuit or the implantable medical device or to perform one or more other functions or transitions, etc. At step 310, one of the control circuit of the implantable medical device or the RFID circuit can enable the communication circuit of the implantable medical device to communicate with the remote device, such as to enable communication between the communication circuit of the implantable medical device and a corresponding communication circuit of the remote device (enabling inquiry or discovery, handshaking, pairing, authentication, encryption, or otherwise establishing secure communication, such as using Bluetooth®, etc.). In certain examples, the configurable memory of the RFID circuit can perform at least a portion of or all data exchange associated with handshaking between the remote device and the communication circuit of the implantable medical device before data is exchanged or in certain examples before the communication circuit of the implantable medical device is enabled. At step 311, one of the control circuit of the implantable medical device or the RFID circuit can enable programming or reprogramming the implantable medical device, such as through communication with the remote device, etc.

Although illustrated as a series of steps, in certain examples, one or more steps can be omitted or be optional, such as the first confirmation at step 304, etc. For example, instead of receiving the first sequence at step 303 for confirmation at step 304, process can proceed from wake up at step 302 to step 305, where one of the control circuit of the implantable medical device or the RFID circuit can provide the second sequence in the second portion of the memory circuit of the RFID circuit, leaving the remote device to request authorization from the authorization circuit with information from the remote device and the second sequence, which can include information about the implantable medical device. In other examples, different combinations or permutations of these or other steps or examples can be combined to form other methods or processes, which is also applicable to other examples discussed herein.

In certain examples, additional security can be implemented in addition to or separate from requiring proximity between the RFID circuit and the remote device. For example, the RFID communication (e.g., near-field communication (NFC), etc.) described herein can include encrypted RFID communication. In other examples, separate from or in addition to encryption, one or more hardware keys can be embedded or otherwise coupled to the remote device. In other examples, separate from or in addition to encryption or implementation of hardware keys, initialization of the patient-specific RFID communication protocol described herein can require that proximity between the RFID communication and the remote device be maintained during initialization of the patient-specific RFID communication protocol. In certain examples, a loss of proximity between the RFID circuit and the remote device (e.g., determined by a detected magnetic field, the RF signal emitted by the remote device, energy from the electromagnetic field emitted by the remote device, etc., falling below a threshold) can trigger generation of a new initialization key, and in certain examples writing the new initialization key to the first portion of the memory circuit of the RFID circuit, such that if a previous initialization key is used to request the authentication sequence from the authentication circuit, and the provided authentication sequence is at least in part based upon the previous initialization key, such provided authentication sequence based on the previous initialization key may not provide authorization or permission with respect to the new, current initialization key of the RFID circuit or the implantable medical device, even if the authentication circuit intended for the remote device to be authorized or permitted for communication with the implantable medical device before the loss of proximity.

In other examples, the memory of the RFID circuit can be used to upload or download other information to or from the implantable medical device, such as connection information, updates, etc., in certain examples additionally requiring one or more aspects of the systems and methods described herein. For example, connection to the communication circuit can be negotiated or set up using the first and second portions of the memory circuit of the RFID circuit, or one or more other updates to the implantable medical device or one or more other components of or coupled to the implantable medical device can be communicated to the implantable medical device using the first and second portions of the memory circuit of the RFID circuit.

Although described herein as having first and second portions, the configurable memory of the memory circuit of the RFID circuit can include additional portions or sub-portions, etc., although at additional cost and in certain examples power or complexity. Accordingly, there are specific advantages of the configurable memory of the RFID circuit only having the first and second portions. However, in other examples, it can be advantageous in other ways to provide additional portions.

The systems and methods described herein provide low-power secure remote reprogramming of implantable medical devices that ensures data can only be written to the implantable medical device or the RFID circuit coupled to the implantable medical device with patient authorization (physical proximity) using a patient-specific RFID communication protocol authorized additionally by a clinician or caregiver associated with the implantable medical device. The systems and methods described herein are designed to integrate seamlessly into existing patient care models and offers significant improvement in the responsiveness of patient-controlled care and reduction in power consumption over existing implantable medical devices and communication protocols.

### Additional Authentication Mechanisms

In other examples, such as in addition to or separate from one or more examples described herein, the systems and methods described herein, including the RFID circuit or separate therefrom, can implement other or additional authentication mechanisms, such as using the authentication circuit, to enhance security of remote programming of implantable medical devices. For example, before programming or reprogramming the implantable medical device, multi-factor authentication can be required for a clinician making programming or reprogramming instructions, in addition to logging into a medical device programmer. In certain examples, such multi-factor authentication can be implemented through email, text, or other authentication action executed using a second remote device of the user seeking authentication, such using a remote or mobile device of the clinician separate from the medical device programmer and controlled or managed by the authentication circuit or one or more other circuits.

For example, a remote device can implement multi-factor authentication requiring both login credentials and a secondary verification code before enabling programming capabilities. After receiving initial login credentials from a clinician making programming changes through an input device, such as of a medical device programmer, etc., the remote device can transmit a verification code to a secondary device associated with the authorized clinician using a network interface device and communication network. In certain examples, the remote device can require entry of this verification code through the input device before proceeding with programming of the implantable medical device.

In an example, in addition to or separate from one or more examples described herein, additional approval can be required from the patient or a caregiver of or associated with the patient (e.g., a family member, a caregiver having a medical or health care power of attorney, a personal care clinician, etc.). For example, a programming or reprogramming instruction for the implantable medical device can be made or received, but prior to implementation, the patient or caregiver can be required to receive a notification of such programming or reprogramming instruction and provide approval, such that the programming or reprogramming instruction is expected by the patient or caregiver and, in certain examples, that the person making the programming or reprogramming recommendation is known by the patient or caregiver.

For example, an external device can include a user interface configured to receive patient or caregiver confirmation of programming changes before such changes are applied to the implantable medical device. The user interface can display authentication information to the patient or caregiver, including an identity of the remote device requesting programming access and details of proposed programming changes. The patient or caregiver can review this information and provide confirmation through the user interface that the programming request is from a trusted source before the programming changes are enabled.

In certain examples, the systems and methods described herein can implement a dual-authorization or second-level approval requirement where programming or reprogramming instructions must be approved by two separate authorized users, for example, two clinicians associated with the patient, etc. For example, a first user can initiate the programming request through the remote device, providing authorization through login credentials and verification. In an example, an authorization request can be transmitted through a network to a second authorized user (e.g., a second clinician) at a secondary remote device. The second authorized user can provide separate authentication and approval through the secondary remote device before the programming or reprogramming instructions are implemented.

In an example, the authentication circuit can generate an authorization sequence for the RFID circuit only after receiving and validating both the first and second users, or the clinician and the patient or caregiver. This dual-authorization mechanism can provide an additional layer of security by requiring two separate authenticated users to approve programming changes, similar to systems and methods requiring two physical keys held by different users to access secure facilities. An external system can maintain records of all programming authorization requests, including the identities of authorizing users, timestamps, and the specific programming changes approved. These records can be reviewed through the remote device to audit the programming authorization process.

### Operation Modes

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

### Physiologic Information

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiration rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiration rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Composite Indications of Patient Condition

Indications of patient condition can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is a HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

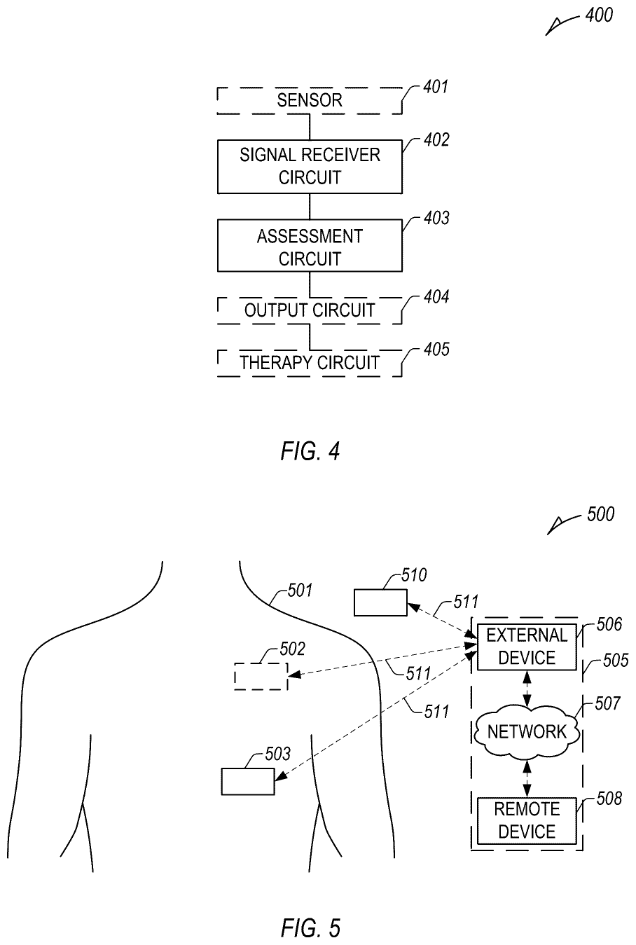

FIG. 4 illustrates an example system 400 (e.g., a medical device system). In an example, one or more aspects of the system 400 can be a component of, or communicatively coupled to, a medical device, such as an implantable medical device (IMD), an insertable cardiac monitor (ICM), an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a body of a patient or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 402 can include the sensor 401. In other examples, the signal receiver circuit can be coupled to or a component of the assessment circuit 403.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

FIG. 5 illustrates an example patient management system 500 and portions of an environment in which the patient management system 500 may operate. The patient management system 500 can perform a range of activities, including remote patient monitoring and diagnosis of a disease condition. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device (AMD), such as an implantable medical device (IMD) 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 502 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include CRT for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming the implantable medical device 502 to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511, such as using one or more communication circuits. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio frequency (RF) telemetry link, or wireless telemetry based on, for example, Bluetooth® or IEEE 802.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The remote device 508 can be configured to evaluate collected patient or patient information and provide alert notifications, among other possible functions. In an example, the remote device 508 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the external device 506 or the remote device 508 can include a respective display unit for displaying the physiologic or functional signals, or alerts, alarms, emergency calls, or other forms of warnings to signal the detection of arrhythmias. In some examples, the external system 505 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 502, the wearable medical device 503, etc., or make additional determinations, etc. Computationally intensive algorithms, such as machine-learning algorithms, can be implemented in the external data processor to process the data retrospectively to detect cardia arrhythmias.

Portions of the one or more ambulatory medical devices or the external system 505 can be implemented using hardware, software, firmware, or combinations thereof. Portions of the one or more ambulatory medical devices or the external system 505 can be implemented using an application-specific or general-purpose control circuit that can be constructed or configured to perform one or more functions. Such a control circuit can include one or more processors, microprocessors, or portions thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of control circuit that can be driven by a code instructing a portion of the control circuit to perform a comparison between the two signals. “Sensors” can include electronic circuits configured to receive information and provide an electronic output representative of such received information.

A therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming the one or more ambulatory medical devices and can receive information about one or more signals acquired by the one or more ambulatory medical devices, such as can be received via a communication link 511. The external system 505 can include a local external implantable medical device programmer. The external system 505 can include a remote patient management system that can monitor patient status or adjust one or more therapies such as from a remote location.

FIG. 6 illustrates a block diagram of an example machine 600 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry, an assessment circuit, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., a data bus). The machine 600 may further include a display unit 610, an input device 612 (e.g., a keyboard), and a user interface (UI) navigation device 614 (e.g., a mouse). In an example, the display unit 610, the input device 612, and the UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 602, the main memory 604, the static memory 606, or the mass storage 608 may be, or include, a machine-readable medium 622 on which is stored one or more sets of data structures or one or more instructions 624 (e.g., software) embodying or utilized by any one or more of the techniques or functions described herein. The one or more instructions 624 may also reside, completely or at least partially, within any of registers of the processor 602, the main memory 604, the static memory 606, or the mass storage 608 during execution thereof by the machine 600. In an example, one or any combination of the processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The one or more instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system comprising:
an implantable medical device comprising:
a control circuit configured to control operation of the implantable medical device;
a communication circuit configured to communicate with a remote device; and
an RFID circuit, separate from the communication circuit of the implantable medical device,
wherein the RFID circuit comprises first and second portions of configurable memory, wherein the RFID circuit is configured to implement a patient-specific RFID communication protocol using the first and second portions of configurable memory,
wherein, to implement the patient-specific RFID communication protocol, the RFID circuit is configured to:
provide an initialization key using the first portion of configurable memory of the RFID circuit; and
receive an authorization sequence from the remote device using the second portion of configurable memory of the RFID circuit,
wherein the implantable medical device or the RFID circuit is configured transition a state of the implantable medical device or the communication circuit of the implantable medical device based on the authorization sequence stored in the second portion of configurable memory of the RFID circuit.

2. The medical device system of claim 1, wherein to transition the state of the implantable medical device includes to enable communication between the remote device and the communication circuit of the implantable medical device based on the authorization sequence stored in the second portion of configurable memory of the RFID circuit.

3. The medical device system of claim 1, wherein the RFID circuit is configured to authenticate the remote device for secure remote programming of the implantable medical device using the first and second portions of configurable memory of the RFID circuit without activating the communication circuit of the implantable medical device.

4. The medical device system of claim 1, wherein the RFID circuit is a passive or semi-passive RFID circuit configured to receive power from the remote device separate from the implantable medical device.

5. The medical device system of claim 1, wherein the implantable medical device includes a power source configured to supply power to the control circuit and the communication circuit,
wherein the RFID circuit is a passive RFID circuit configured to receive power from the remote device separate from the implantable medical device and does not receive power from the power source of the implantable medical device.

6. The medical device system of claim 1, wherein the RFID circuit comprises a memory circuit comprising a limited amount of configurable memory, wherein the limited amount of configurable memory consists of the first and second portions of configurable memory.

7. The medical device system of claim 1, wherein the implantable medical device comprises a data bus configured to enable communication between the communication circuit of the implantable medical device and the control circuit of the implantable medical device,
wherein the RFID circuit comprises an RFID control circuit and an internal data bus between the RFID control circuit and the configurable memory of the RFID circuit,
wherein the RFID circuit comprises an RFID antenna, separate from an antenna of the communication circuit, configured to receive energy to power the internal data bus of the RFID circuit.

8. The medical device system of claim 7, wherein the RFID control circuit is configured to control activation of the data bus between the communication circuit of the implantable medical device and the control circuit of the implantable medical device based on the authorization sequence stored in the second portion of configurable memory of the RFID circuit.

9. The medical device system of claim 1, wherein the initialization key is a random sequence provided by the RFID circuit, wherein the authorization sequence is a patient-specific passkey generated by an authentication circuit separate from the remote device,
wherein the implantable medical device or the RFID circuit is configured to transition the state of the implantable medical device or the communication circuit of the implantable medical device based on the patient-specific passkey generated by the authentication circuit and stored in the second portion of configurable memory of the RFID circuit.

10. The medical device system of claim 1, wherein the implantable medical device is configured transition a state of the communication circuit of the implantable medical device based on the authorization sequence stored in the second portion of configurable memory of the RFID circuit.

11. The medical device system of claim 1, wherein the RFID circuit is configured transition a state of the implantable medical device based on the authorization sequence stored in the second portion of configurable memory of the RFID circuit.

12. The medical device system of claim 1, wherein, to implement the patient-specific RFID communication protocol, the RFID circuit is configured to:
detect, at a first time, a wake-up signal from the remote device;
activate an internal data bus of the RFID circuit;
receive, at a second time subsequent to the first time, an identifier of the remote device using one of the first or second portions of configurable memory of the RFID circuit;
provide, at a third time subsequent to the second time, the initialization key using the first portion of configurable memory of the RFID circuit;
receive, at a fourth time subsequent to the third time, the authorization sequence from the remote device using the second portion of configurable memory of the RFID circuit; and
authenticate, at a fifth time subsequent to the fourth time, the remote device for secure remote programming of the implantable medical device using the communication circuit of the implantable medical device based on the authorization sequence and the initialization key.

13. The medical device system of claim 12, wherein to authenticate the remote device comprises to perform multi-factor authentication of a clinician providing programming instructions for secure remote programming of the implantable medical device, perform second-level approval by a second clinician of the programming instructions for secure remote programming of the implantable medical device, or perform patient approval of the clinician providing programming instructions or programming instructions for secure remote programming of the implantable medical device.

14. A method comprising:
implementing a patient-specific RFID communication protocol using first and second portions of configurable memory of an RFID circuit of an implantable medical device,
wherein the implantable medical device comprises a control circuit configured to control operation of the implantable medical device and a communication circuit configured to communicate with a remote device, wherein the communication circuit is separate from the RFID circuit,
wherein implementing the patient-specific RFID communication protocol comprises:
providing an initialization key using the first portion of configurable memory of the RFID circuit;
receiving an authorization sequence from the remote device using the second portion of configurable memory of the RFID circuit; and
transitioning a state of the implantable medical device or the communication circuit of the implantable medical device based on the authorization sequence in the second portion of configurable memory of the RFID circuit.

15. The method of claim 14, wherein transitioning the state of the implantable medical device includes enabling communication between the remote device and the communication circuit of the implantable medical device based on the authorization sequence stored in the second portion of configurable memory of the RFID circuit.

16. The method of claim 14, comprising:
authenticating the remote device for secure remote programming of the implantable medical device using the first and second portions of configurable memory of the RFID circuit without activating the communication circuit of the implantable medical device.

17. The method of claim 14, comprising:
receiving power from the remote device separate from the implantable medical device to power the RFID circuit,
wherein the RFID circuit is a passive or semi-passive RFID circuit.

18. The method of claim 14, comprising:
supplying power to the control circuit and the communication circuit using a power source of the implantable medical device; and
receiving power from the remote device separate from the implantable medical device to power the RFID circuit,
wherein the RFID circuit is a passive RFID circuit configured that does not receive power from the power source of the implantable medical device.

19. The method of claim 14, wherein the RFID circuit comprises a memory circuit comprising a limited amount of configurable memory, wherein the limited amount of configurable memory consists of the first and second portions of configurable memory.

20. The method of claim 14, comprising:
enabling communication between the communication circuit of the implantable medical device and the control circuit of the implantable medical device using a data bus of the implantable medical device;
enabling communication between an RFID control circuit of the RFID circuit and the configurable memory of the RFID circuit using an internal data bus of the RFID control circuit;
receiving energy to power the internal data bus of the RFID circuit using an RFID antenna of the RFID circuit, separate from an antenna of the communication circuit; and
controlling, using the RFID control circuit, activation of the data bus between the communication circuit of the implantable medical device and the control circuit of the implantable medical device based on the authorization sequence stored in the second portion of configurable memory of the RFID circuit.
